(19) United States
(12) Patent Application Publication
(10) Pub. No.: US 2008/0160604 A1
(43) Pub. Date: Jul. 3, 2008

(54) APPARATUS FOR PRODUCING A STABLE OXIDIZING BIOCIDE

(76) Inventors: Amit Gupta, Aurora, IL (US); Manian Ramesh, Barrington Hill, IL (US); Randall Elliott, Naperville, IL (US)

Correspondence Address: NALCO COMPANY, 1601 W. DIEHL ROAD, NAPERVILLE, IL, 60563-1198, US

(21) Appl. No.: 11/618,174

(22) Filed: Dec. 29, 2006

(51) Int. Cl. C12M 1/06; C12M 1/04

(52) U.S. Cl. 435/289.1

(57) ABSTRACT

The invention relates to an apparatus for the production of oxidizing biocides. The apparatus can be used as a remote production system where the product is stored use or it can be an in situ production system where the product can be in fluid connection with the system being treated. The preferred product of the system is chloramine, which can be produced in a stable form with this apparatus.

### COPYRIGHT NOTICE

A portion of the disclosure of this patent document contains or may contain copyright protected material. The copyright owner has no objection to the photocopy reproduction by anyone of the patent document or the patent disclosure in exactly the form it appears in the Patent and Trademark Office patent file or records, but otherwise reserves all copyright rights whatsoever.

### TECHNICAL FIELD

This invention relates to the production of a stable oxidizing biocide and the apparatus used in the said production. The invention has several embodiments of the apparatus but there are two main embodiments of the invention, in situ production and remote production. The fact that the oxidizing biocide is in a more stable form allows for its production, storage and transportation. The invention demonstrates the apparatus used to produce a stable and functional chloramine as one example of a stable oxidizing biocide, which allows for the use of chloramines in water treatment systems, and a wide variety of other treatment systems, as biocidal composition without its rapid degradation.

### BACKGROUND

The invention described here pertains to the apparatus for production of a biofouling control agent. The basis for the invention is an apparatus that provides for the composition of the reactants and the conditions for production using concentrated reactants, to convert two liquid solutions from their native chemical form to another, with altered biocidal properties.

Throughout the world, there are many different types of industrial water systems. Industrial water systems exist so that necessary chemical, mechanical and biological processes can be conducted to reach the desired outcome. Fouling can occur even in industrial water systems treated with the best water treatment programs currently available. For purposes of this patent application “fouling” is defined as “the deposition of any organic or inorganic material on a surface”.

If these industrial water systems are not treated for microbial fouling control, then they will become heavily fouled. Fouling has a negative impact on the industrial water system. For example, severe mineral scale (inorganic material) will buildup on the water contact surfaces and anywhere there is scale, there is an ideal environment for the growth of microorganisms.

Fouling occurs by a variety of mechanisms including deposition of air-borne and water-borne and water-formed contaminants, water stagnation, process leaks, and other factors. If allowed to progress, the system can suffer from decreased operational efficiency, premature equipment failure, loss in productivity, loss in product quality, and increased health-related risks associated with microbial fouling.

Fouling can also occur due to microbiological contamination. Sources of microbial contamination in industrial water systems are numerous and may include, but are not limited to, air-borne contamination, water make-up, process leaks and improperly cleaned equipment. These microorganisms can establish microbial communities on any wetted or semi-wetted surface of the water system. Once these microbial populations are present in the bulk water more than 99% of the microbes present in the water will be present on all surfaces, in the form of biofilms.

Exopolymeric substance secreted from the microorganisms aid in the formation of biofilms as the microbial communities develop on the surface. These biofilms are complex ecosystems that establish a means for concentrating nutrients and offer protection for growth. Biofilms can accelerate scale, corrosion, and other fouling processes. Not only do biofilms contribute to reduction of system efficiencies, but they also provide an excellent environment for microbial proliferation that can include pathogenic bacteria. It is therefore important that biofilms and other fouling processes be reduced to the greatest extent possible to maximize process efficiency and minimize the health-related risks from water-borne pathogens.

Several factors contribute to the problem of biological fouling and govern its extent. Water temperature; water pH; organic and inorganic nutrients, growth conditions such as aerobic or anaerobic conditions, and in some cases the presence or absence of sunlight, etc. can play an important role. These factors also help in deciding what types of microorganisms might be present in the water system.

As described earlier, biological fouling can cause unwanted process interferences and therefore must be controlled. Many different approaches are utilized for the control of biological fouling in industrial processes. The most commonly used method is the application of biocidal compounds to the process waters. The biocides applied may be oxidizing or non-oxidizing in nature. Due to several different factors such as economics and environmental concerns, the oxidizing biocides are preferred. Oxidizing biocides such as chlorine gas, hypochlorous acid, bromine derived biocides, and other oxidizing biocides are widely used in the treatment of industrial water systems.

One factor in establishing the efficacy of oxidizing biocides is the presence of components within the water matrix that would constitute a “chlorine demand” or oxidizing biocide demand. “Chlorine demand” is defined as the quantity of chlorine that is reduced or otherwise transformed to inert forms of chlorine by substances in the water. Chlorine-consuming substances include, but are not limited to, microorganisms, organic molecules, ammonia and amino derivatives; sulfides, cyanides, oxidizable cations, pulp lignins, starch, sugars, oil, water treatment additives like scale and corrosion inhibitors, etc. Microbial growth in the water and in biofilms contributes to the chlorine demand of the water and to the chlorine demand of the system to be treated. Conventional oxidizing biocides were found to be ineffective in waters containing a high chlorine demand, including heavy slimes. Non-oxidizing biocides are usually recommended for such waters.

Chloramines are effective and are typically used in conditions where a high demand for oxidizing biocides such as chlorine exists or under conditions that benefit from the persistence of an ‘oxidizing’ biocide. Domestic water systems are increasingly being treated with chloramines. Chloramines are generally formed when free chlorine reacts with ammonia present or added to the waters. Many different methods for production of chloramines have been documented. Certain key parameters of the reaction between the chlorine and the nitrogen source determine the stability, and efficacy of the produced biocidal compound. The previously described methods have relied on either the pre-formation of dilute solutions of the reactants followed by their combination to produce a solution of chloramines. The reactants are an amine source in the form of an ammonium salt (sulfate, bromide, or chloride) and a Cl-donor (chlorine donor) in the form of gas or combined with alkali earth metal (Na or Ca). Also, the described methods have relied on controlling the pH of the reaction mix by addition of a reactant at a high pH or by the separate addition of a caustic solution. The disinfectant thus produced must be immediately fed into the system being treated since the disinfectant degrades rapidly. The disinfectant solution is generated outside the system being treated and then fed into the aqueous system for treatment. In previously described methods of production for treatment of liquids to control biological fouling, a significant problem occurred in that the active biocidal ingredient was unstable chemically and rapidly decomposed with a resulting fast drop in pH. This rapid deterioration of the biocidal ingredient resulted in a loss in efficacy. It was also observed that the pH of the active biocidal ingredient was never >8.0 due to the rapid decomposition of the biocidal component (referenced in U.S. Pat. No. 5,976,386).

### SUMMARY

The current invention describes the following key aspects:
1. An apparatus to properly prepare a stable oxidizing biocide.
2. An apparatus, which is able to produce an oxidizing biocide either as remote production or in situ production.
3. A method for producing a stable oxidizing biocide without the need to dilute the reaction components prior to combination.
4. The stable oxidizing biocide produced is chloramine.

### BRIEF DESCRIPTION OF THE FIGURES

FIG. 1 a schematic view of one embodiment of the apparatus.

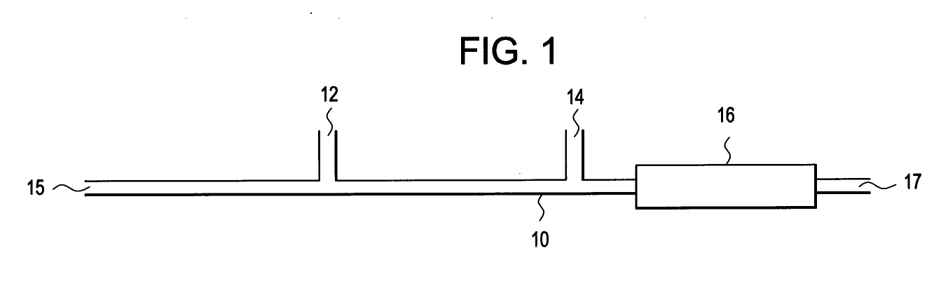

FIG. 2 a schematic view of another embodiment of the apparatus.

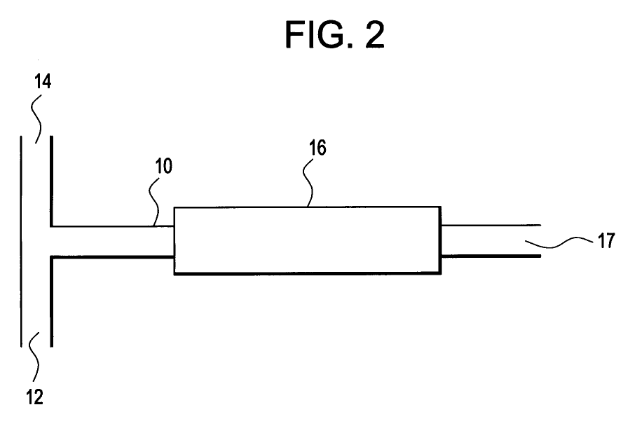

### DETAILED DESCRIPTION

The foregoing may be better understood by reference to the following figures, which are intended to illustrate methods for carrying out the invention and are not intended to limit the scope of the invention.

The invention relates to an apparatus for the production of a stable oxidizing biocide 10 comprising, a first feed line 12, a second feed line 14, a third feed line 15, an agitator 16, and a product outlet 17. The third feed line 15 of the invention is used to supply the reaction means and reactants into the apparatus to produce the stable oxidizing biocide. The third feed line 15 is used for the reaction means, which is preferably water and most preferably the drive water of the system. The drive water can be derived from the process being treated with the oxidizing biocide.

In another embodiment of the invention the apparatus 10 for the production of a stable oxidizing biocide comprising, a first feed line 12, a second feed line 14, an agitator 16, and a product outlet 17. The first 12 and second feed lines 14 are for the transport of the reactants that are used to produce the stable oxidizing biocide. The embodiments of the present invention contain the following components in common therefore the description below is relevant to all embodiments.

The agitator 16 of the invention preferably is an inline mixer that is most preferably static. The product outlet 17 of the invention is directly connected to the process being treated to provide in situ production of the oxidizing biocide or may be in connection with a storage device to store the oxidizing biocide for later use. The invention also can have the product outlet 17 in fluid connection with the process being treated to provide in situ production of the oxidizing biocide.

The preferred stable biocide for production with the apparatus 10 is stable chloramine. The reactants that pass through the first 12 and second feed lines 14 for the production of stable chloramine are concentrated chlorine source and concentrated amine source.

It should be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. An apparatus for the production of a stable oxidizing biocide comprising:
(i) a first feed line,
(ii) a second feed line,
(iii) a third feed line,
(iv) an agitator, and
(v) a product outlet.

2. The apparatus of claim 1 wherein the first, second and third feed lines transport the reaction means and reactants to produce the stable oxidizing biocide.

3. The apparatus of claim 1 wherein the third feed line is used for the reaction means.

4. The apparatus of claim 3 wherein the reaction means is water.

5. The apparatus of claim 3 wherein the reaction means is also the drive water of the system.

6. The apparatus of claim 5 where in the drive water is derived from the process being treated with the oxidizing biocide.

7. The apparatus of claim 1 wherein the agitator is an inline mixer.

8. The apparatus of claim 7 wherein the inline mixer is static.

9. The apparatus of claim 6 wherein the product outlet is directly connected to the process being treated to provide in situ production of the oxidizing biocide.

10. The apparatus of claim 5 wherein the product outlet is in fluid connection with a storage device.

11. The apparatus of claim 5 wherein the product outlet is in fluid connection with the process being treated to provide in situ production of the oxidizing biocide.

12. The apparatus of claim 2 wherein the stable oxidizing biocide is chloramine.

13. The apparatus of claim 12 wherein the reactants are a concentrated chlorine source and a concentrated amine source.

14. An apparatus for the production of a stable chloramine comprising:
(i) a first feed line,
(ii) a second feed line,
(iii) a third feed line,
(iv) an agitator, and
(v) a stable chloramine outlet.

15. The apparatus of claim 14 wherein the first, second and third feed lines are used to supply the reaction means, the Chlorine source and the amine source to produce the stable chloramine.

16. The apparatus of claim 15 where in the Chlorine source and the amine source are used in concentrated form.

17. The apparatus of claim 14 wherein the third feed line is used for the reaction means.

18. The apparatus of claim 17 wherein the reaction means is water.

19. The apparatus of claim 17 wherein the reaction means is also the drive water of the system.

20. The apparatus of claim 19 where in the drive water is derived from the process being treated with stable chloramine.

21. The apparatus of claim 14 wherein the agitator is an inline mixer.

22. The apparatus of claim 21 wherein the inline mixer is static.

23. The apparatus of claim 20 wherein the product outlet is directly connected to the process being treated to provide in situ production of the stable chloramine.

24. The apparatus of claim 19 wherein the product outlet is in fluid connection with a storage device.

25. The apparatus of claim 19 wherein the product outlet is in fluid connection with the process being treated to provide in situ production of the stable chloramine.

26. An apparatus for the production of a stable oxidizing biocide comprising:
(i) a first feed line,
(ii) a second feed line,
(iii) an agitator, and
(iv) a stable oxidizing biocide outlet.

27. The apparatus of claim 26 wherein the first and second feed lines are used to supply the reactants for production of the stable oxidizing biocide.

28. The apparatus of claim 27 where in the reactants are used in concentrated form.

29. The apparatus of claim 26 wherein the agitator is an inline mixer.

30. The apparatus of claim 29 wherein the inline mixer is static.

31. The apparatus of claim 29 wherein the product outlet is in fluid connection with a storage device.

32. The apparatus of claim 29 wherein the product outlet is in fluid connection with the process being treated.

33. The apparatus of claim 32 wherein the stable oxidizing biocide is chloramine.

34. The apparatus of claim 33 wherein the reactants are a concentrated chlorine source and a concentrated amine source.
